(19) United States
(12) Patent Application Publication
(10) Pub. No.: US 2026/0041568 A1
(43) Pub. Date: Feb. 12, 2026

(54) PROTECTIVE PROSTHETIC FOOT SHELL COVER

(71) Applicant: Faezeh AZHARI, Toronto (CA)

(72) Inventor: Faezeh AZHARI, Toronto (CA)

(21) Appl. No.: 19/292,725

(22) Filed: Aug. 6, 2025

Related U.S. Application Data
(60) Provisional application No. 63/680,339, filed on Aug. 7, 2024.

(51) Int. Cl. A61F 2/78; A61F 2/50

(52) U.S. Cl. CPC A61F 2/78; A61F 2002/5001; A61F 2002/7881

(57) ABSTRACT

A prosthetic foot shell cover includes a stirrup, a top portion and an elastic rim. The prosthetic foot shell cover is for used with a prosthetic foot assembly including a foot shell (having heel, an arch and a back), a prosthetic foot and ankle, and a pylon operably attached to the foot shell. The stirrup is configured to go under the foot shell. The top portion is integrally connected to the stirrup and the top portion is configured to extend over the arch and around the back of the prosthetic foot. The elastic rim is integrally attached to the top portion and the elastic rim is configured to engage the pylon of the prosthetic foot. The stirrup, top portion and elastic rim are a unitary construction of resiliently deformable material. Two seals are required to achieve water resistance or water tightness, one at the foot shell and one at the pylon or ankle component.

### BACKGROUND

The present disclosure relates to the protection of prosthetic feet from water, chemicals, snow, dust, sand, rocks, leaves, and other debris or harsh materials by covering the opening at the top of the prosthetic foot shell. More specifically, this may be an elastic slip-on, waterproof, disposable or semi-disposable foot shell cover. The shell cover is configured to prevent the ingress of liquids and debris.

Prosthetic feet are typically covered by a foot shell that resembles a human foot. The top of the foot shell, however, is open, which allows dirt, dust, sand, debris, snow, and water to enter the shell and collect there. This leads to multiple problems, including but not limited to 1) malfunction and wear/corrosion of the prosthetic foot components; 2) difficulty cleaning the shell and foot; 3) difficulty swimming or showering due to the added weight of water collected in the shell; and 4) safety issues when water or debris are spilled out causing slippery or obstructed surfaces.

Therefore, it is sometimes necessary to cover the top of the foot shell to avoid these problems. Some of the methods prosthesis users often use to achieve this include using duct tape or cast covers at the beach or when showering (to prevent water and sand from entering the foot shell); using nylon socks when hiking (to prevent debris entering); or creating holes in the foot shell to drain water. These solutions are often ineffective and/or unsightly.

Accordingly, it would be advantageous to provide a device that solves the problem of water and debris entrance and collection in the foot shell. In addition, it would be advantageous to provide a design that provides a device that may secure the foot shell to sandals and flip flops in a straightforward and seemly manner. Since sandals and flip flops are often worn at beaches and waterparks where sand and water can enter the foot shell, such a device or devices would be useful to prosthesis users.

### SUMMARY

A prosthetic foot shell cover includes a stirrup, a top portion and an elastic rim. The prosthetic foot shell cover is used with a prosthetic foot assembly including a foot shell, a prosthetic foot, optional ankle components, and a pylon that is operably attached to the foot assembly. The foot shell has a heel, an arch and a back. The stirrup is configured to go under the prosthetic foot shell. The top portion is integrally connected to the stirrup and the top portion is configured to extend over the arch and around the back of the prosthetic foot shell. The elastic rim is integrally attached to the top portion and the elastic rim is configured to engage the pylon of the prosthetic foot assembly. The stirrup, top portion and elastic rim are a unitary construction of resiliently deformable material. In use, the foot shell cover provides two seals, one at the foot shell level and the other at the pylon or ankle component.

The protective prosthetic foot shell cover may be made from elastic latex. The resiliently deformable material of the protective prosthetic foot shell cover may be made from silicone, neoprene or other suitable material.

The top portion of the protective prosthetic foot shell cover may further include a bulbous extension and the elastic rim is integrally attached to the bulbous extension. The elastic rim may be a rolled seal. The elastic rim may be a flat seal. The foot shell may include a heel attached to the stirrup.

The prosthetic foot shell has a space between the big toe and a little toe and the foot shell further includes a thong segment integrally attached to the stirrup and the thong segment is configured to be positioned in the space between the big toe and the little toe of the foot shell. The shell cover may be for use with a prosthetic foot including the foot shell and a thong or sandal having a hook and loop type fastener on the top thereof and the thong segment may further include a thong segment hook and loop type fastener attached thereto proximate to the stirrup and the thong segment hook and loop type fastener is configured to engage with the hook and loop type fastener on the thong.

The protective prosthetic foot shell cover may further include a sandal portion integrally attached to the stirrup and configured to be spaced from top portion. The protective prosthetic foot shell cover may be for use with a prosthetic foot including the foot shell and a sandal having at least one hook and loop type fastener attached thereto and wherein the sandal portion further includes sandal at least one hook and loop type fastener attached to sandal portion and the sandal hook and loop type fastener is configured to engage with the hook and loop type fastener on the sandal. The sandal may include a plurality of hook and look type fasteners and the sandal portion includes a plurality of hook and loop types fasteners. The top portion of the protective prosthetic foot shell cover may further include a plurality of links for receiving a tie. The protective prosthetic foot shell cover of claim 12 wherein the tie is tied to a shoe. The protective prosthetic foot shell cover may be customizable.

### BRIEF DESCRIPTION OF THE DRAWINGS

Embodiments will now be described, by way of example only, with reference to the drawings, in which:

FIG. 1A shows a perspective view of a protective prosthetic foot shell cover in use on a prosthetic leg;

FIG. 1B is a photograph showing the elements of a prosthetic foot assembly;

FIG. 2A is an enlarged perspective view of the protective prosthetic foot shell cover and the prosthetic foot assembly portion of the prosthetic leg shown in FIG. 1A;

FIG. 2B is an enlarged perspective view of the protective prosthetic foot shell cover of FIG. 1A but shown with a prosthetic leg that includes a bulbous top end;

FIG. 2C is an enlarged perspective view of the protective prosthetic foot shell cover of FIG. 1A but shown with a prosthetic leg that includes a flat seal at the top end;

FIG. 2D is an enlarged perspective view of the protective prosthetic foot shell cover similar to that shown in FIG. 2A but also including a heel portion.

FIG. 3A is an enlarged perspective view of an alternate configuration of a protective prosthetic foot shell cover similar to that shown in FIG. 1A but configured for use with thong type sandal;

FIG. 3B is a separated view showing a bottom view of the protective prosthetic foot shell cover of FIG. 3A and showing a top view of the thong type sandal;

FIG. 4A is an enlarged perspective view of an alternate configuration of a protective prosthetic foot shell cover similar to that shown in FIG. 1A but configured for use with slide type sandal;

FIG. 4B is a separated view showing a bottom view of the protective prosthetic foot shell cover of FIG. 4A and showing a top view of the slide type sandal; and

FIG. 5 is a separated view showing a side view of an alternate configuration of a protective prosthetic foot shell cover similar to that shown in FIG. 1A but configured for use with a cross over slide type sandal and a top view of the cross over slide type sandal.

### DETAILED DESCRIPTION

A further understanding of the functional and advantageous aspects of the shell cover can be realized by reference to the following detailed description and drawings.

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein the “operably connected” or “operably attached” means that the two elements are connected or attached either directly or indirectly. Accordingly, the items need not be directly connected or attached but may have other items connected or attached therebetween.

The following examples are presented to enable those skilled in the art to understand and to practice embodiments of the present disclosure. They should not be considered as a limitation on the scope of the disclosure, but merely as being illustrative and representative thereof.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

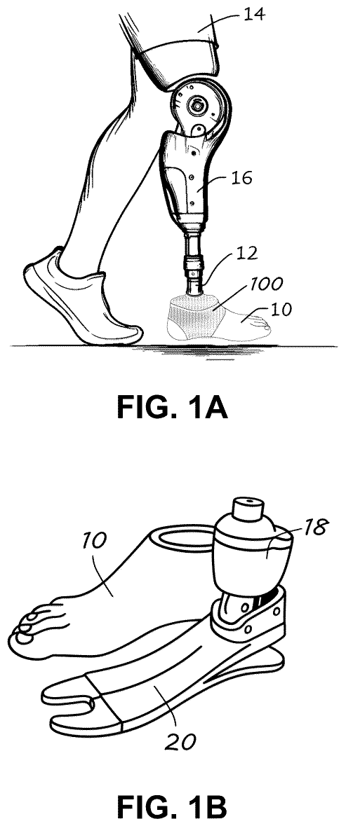

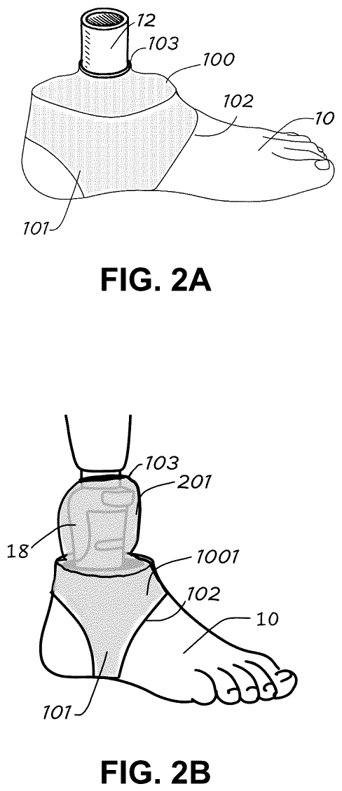

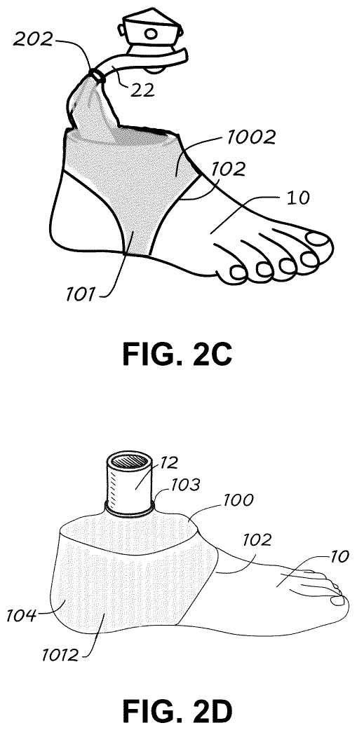

Referring to FIG. 1A, a prosthetic foot shell cover that is a single, seamless piece of thin resiliently deformable material that may be but not limited to latex or silicone or other polymer material is shown generally at 100. The foot shell cover 100 is for use with a prosthetic foot assembly. As shown in FIG. 1B the prosthetic foot assembly includes a prosthetic foot shell, a prosthetic foot. The prosthetic foot assembly may include an ankle component as shown herein. A pylon that is operably attached to the foot assembly. The prosthetic foot shell has a heel, an arch and a back. The foot shell cover 100 has a stirrup 101 at the bottom and the top 102 is open with a small cross-section similar to a balloon opening that stretches around the pylon, providing a seal. The top part may be bulbously 201 shaped to fit larger foot-to-pylon attachments or different ankle components as shown in FIG. 2B. The stirrup stretches over the foot shell to provide a close seal.

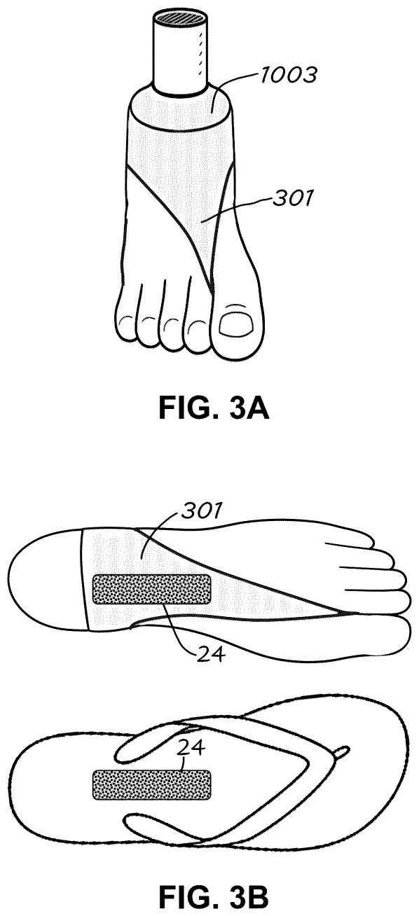

The shell cover can be either just a simple stirrup or extend to cover other parts of the foot, providing functionality with footwear as shown in FIGS. 3A to 5. In use, the shell cover can be simply stretched at the top and stirrup, slipped onto the foot shell, and stretched to fit such that the stirrup is in place, conforming to the shape of the foot shell. The foot shell opening at the top is covered and secured/sealed over the pylon or other component as appropriate such that water or debris cannot enter the foot shell, thereby protecting the prosthetic foot components and allowing for the user's functional activity. The cover can be worn for extended periods of time and replaced once it is dirty or loses elasticity.

FIG. 1A is a side view of the shell cover in its simplest form as worn over a prosthetic foot shell assembly 10 and operably secured at the pylon 12. This figure also shows the socket 14 and the prosthetic components 16 (e.g., knee+pylon+foot assembly). The foot shell has a heel, an arch and a back.

FIG. 1B shows the different components of the foot assembly: the prosthetic foot 20, ankle 18, and foot shell 10. It will be appreciated by those skilled in the art the prosthetic components 16 and foot shell 10 are shown by way of example and that there are a number of variations in the shapes and configurations thereof.

FIG. 2A is a close-up view of the shell cover in its simplest form 100 as worn over a prosthetic foot shell 10 and secured at the pylon. The protective shell cover has a stirrup 101 at the bottom and allows for an elasticity seal 102 to the foot shell 10 and provides a seal with the foot shell. The top end stretches over the opening and ends in an elastic rim (tube-like opening that may be rolled at the top) 103 with a small cross-section that stretches to seal to the pylon 12, providing a secure closure.

To wear, the narrower top end 103 is widened and aligned with the stirrup openings, and the prosthesis user inserts their prosthetic foot into the stretched opening and slips the cover on so that the stirrup is in place and the area around the opening is fully covered and stretched to seal. Then, the narrow top end 103 is released to snugly secure the cover to the pylon (or another component as appropriate).

FIG. 2B shows an alternative embodiment 1001 with a bulbous top end 201 to accommodate larger ankle components 18 or small blades. The bulbous top end may be an elongate bulbous top end that extends over a prosthetic knee.

FIG. 2C shows an alternative embodiment 1002 with a longer top end that has a flat seal (not a rolled ring shape) 202 for blades 22 or other/larger components.

FIG. 2D shows an alternative embodiment 1012 that is similar to the simplest form 100 shown in FIG. 2A but further including a heel portion 104 that is attached to the stirrup 102. Shell cover 1012 may be formed as a unitary piece.

Another problem users of prosthetic feet suffer from is wearing sandals and flip flops with no back. Due to the lack of muscle control, these footwears often fall off and/or rotate and cause gait problems and falls. Amputees often attach their foot shell to the footwear using different types of fasteners such as but not limited to a generic hook and loop type fasteners, Velcro™, snap buttons, or elastic bands/straps. These attachments and contraptions, if on the foot shell, must be removed/replaced often, and if on the footwear, need to be made specifically for the footwear and can be unsightly. Other embodiments of the foot shell cover can be designed to include segments or links for securing footwear. Since sandals and flip flops are often worn at beaches and waterparks, the prosthesis user can use both functionalities if needed. These additional segments can be either of the same material and connected seamlessly with the basic form or of a different material (e.g., fabric) attached to the basic form such that the entire foot shell cover is a unitary piece. Other designs may include these additions separately such that the user is able to secure them to the basic form.

FIG. 3A and FIG. 3B show the top and bottom views, respectively, of an example embodiment 1003 with an additional ‘thong’ segment 301, which can be furnished with a fastener 24 including but not limited to adhesives, generic hoor and loop type fastener, e.g., Velcro™ or snap buttons for securing the foot shell to flip flops or another footwear.

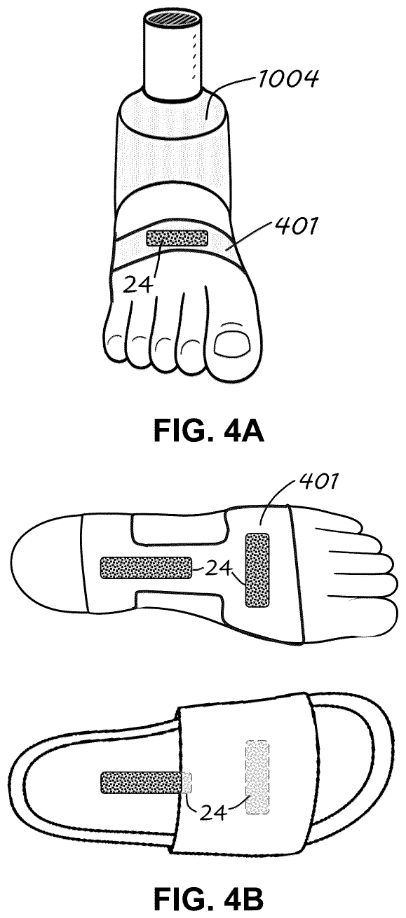

FIG. 4A and FIG. 4B show the top and bottom views, respectively, of an example embodiment 1004 with an additional ‘sandal’ segment 401, which can be furnished with a fastener 24 such as adhesives generic hook and loop type fasteners, Velcro™ or snap buttons for securing the foot shell to sandals or another footwear.

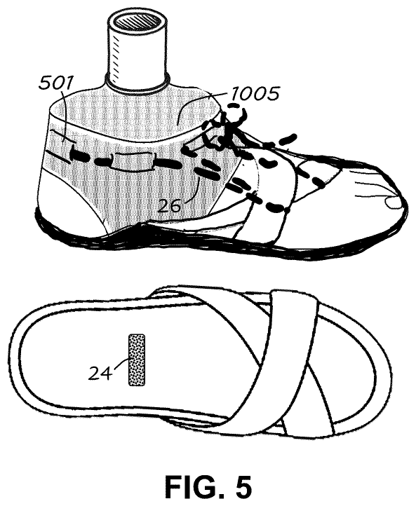

FIG. 5 shows the side view of an example embodiment 1004 with links 106 to accommodate ties or straps 26 for securing footwear. These may be links or double layer segments.

As discussed above the foot shell cover is configured to provide a seal around the foot shell and one of the pylon and ankle component. More specifically there is one seal at the top end to the pylon or ankle component and another seal at the foot shell. The seal around the foot shell may be achieved by the foot shell cover being tight enough around a part of the foot shell to create a complete seal. As can be seen in the various examples shown in the figures the shapes and seals can be different depending on the prosthetic foot components and the foot shell shape.

It will be appreciated that there are variations of the foot shell cover that may be included in the design. For example, the stirrup 101 may be configured in two parts that are attachable. This may make it easier for some users to don and doff. In such an embodiment the user would pull on the foot shell cover and then connect the two parts of the stirrup.

It will be appreciated that the foot shell cover over time might degrade over time, it might lose its ability to seal or it might get dirty. Accordingly, the foot shell cover is designed to be disposable in those scenarios. However, it is anticipated that it may be used multiple times before disposal.

The invention embodiment descriptions were provided for illustration purposes and are not intended to be exhaustive or to limit the invention embodiments. Many modifications and variations are possible and may be evident to prosthesis users, prosthetists, and other persons skilled in the art. The following claims intend to include all such embodiments within the scope of this invention:

A method of protecting prosthetic feet components through a foot shell cover as a single, seamless thin piece of silicone, latex or polymer material with a bottom stirrup and a bulbous top end with a small opening that seals around the pylon (or other prosthetic component at the top of the foot as appropriate). The cover may be reusable, disposable or semi-disposable. In use, the cover is slipped onto the foot shell and pulled/stretched over to the heel such that the stirrup secured the cover at the heel and the open sides naturally seal to the foot shell material when stretched. The top part stretches over the foot shell opening, conforms to any ankle components, and is generally sealed perfectly to the pylon at the top end.

The shell cover is made from a resiliently deformable material such as polymer or fabric and is thin, flexible, and elastic/stretchable to permit easy donning and doffing.

The material may be waterproof and may be latex, silicone or neoprene fabric. The material is as thick or slightly thicker than a standard latex balloon. The size and shape can be customized to different foot shell sizes. The size and shape of the stirrup opening can be customized to different foot shell sizes and coverage. The size and shape of the top part can be customized to accommodate different ankle/foot components. Accordingly, the shell cover is customizable.

The size and form of the top rim/seal can vary depending on the components it is sealing to (e.g., pylon vs blade). The top rim may be rolled or flat and/or reinforced (double layer). The open sections to be sealed can be reinforced and have a layer of self-fusing silicone (or other tacky material) for a better seal to the foot shell.

The protective cover may have a variety of shapes (or additional segments) at the bottom or top (see other embodiments) and be produced in a variety of colors (including transparent) or patterns.

The protective cover may be disposable or semi-disposable as depending on wear and tear and exposure to harsh environments, it may be reused many times or kept on for a long time (since it does not interfere in any way with the functionality of the prosthesis and foot shell. Once worn out or dirty, it can be easily replaced.

The protective cover is simple to don and doff with no fasteners or closures and does not fall off as it is stretched over the foot shell and is anchored to the bottom using the stirrup.

The protective cover can have the basic embodiment or have additional segments/links/accessories for easy securing or attachment to footwear.

The basic embodiment of the foot shell cover is made in one-piece and is seamless.

The shell cover can be manufactured in a similar manner as balloons or latex gloves or other well-known methods. For example, it can be made in a closed form (like a balloon) followed by cutting the required openings and reinforcing the opening edges.

The shell cover has a universal design adapted to fit most and ideally all feet and most build heights. The shell cover is simple and elegant. The coverage by the shell cover of the foot shell can vary. The shell cover may have various colors or patterns (e.g., transparent, tattoo, etc.)

The shell cover typically takes seconds to don and doff. In use, it requires no functional change to the user's prosthetic foot, gait, joint, etc.

The shell cover may be used with footwear: 1) Velcro™ or other attachment to bottom; 2) accommodate straps to secure footwear.

1. A prosthetic foot shell cover for use with a prosthetic foot assembly including a foot shell (having heel, an arch and a back), a prosthetic foot and ankle, and a pylon that is operably attached to the foot assembly, the shell cover comprising:
a stirrup configured to go under the prosthetic foot shell;
a top portion integrally connected to the stirrup, the top portion configured to extend over the arch and around the back of the prosthetic foot shell; and
an elastic rim integrally attached to the top portion, the elastic rim configured to engage the pylon attached to the prosthetic foot assembly;
whereby the stirrup, top portion and elastic rim being a unitary construction of resiliently deformable material and configured to provide a seal around the foot shell and one of the pylon and ankle component.

2. The protective prosthetic foot shell cover of claim 1 wherein the shell cover is made from elastic latex.

3. The protective prosthetic foot shell cover of claim 1 wherein the shell cover is made from silicone.

4. The protective prosthetic foot shell cover of claim 1 wherein the top portion further includes a bulbous extension and the elastic rim is integrally attached to the bulbous extension.

5. The protective prosthetic foot shell cover of claim 1 wherein the elastic rim is a rolled seal.

6. The protective prosthetic foot shell cover of claim 1 wherein the elastic rim is a flat seal.

7. The protective prosthetic foot shell cover of claim 1 further including a heel portion connected to the stirrup.

8. The protective prosthetic foot shell cover of claim 1 wherein the prosthetic foot shell has a space between a big toe and a little toe and the foot shell further including a thong segment integrally attached to the stirrup and the thong segment is configured to be positioned in the space between the big toe and the little toe of the prosthetic foot shell.

9. The protective prosthetic foot shell cover of claim 8 wherein the shell cover is for use with a thong as well as foot shell, the thong having a fastener on the top thereof and wherein the thong segment further includes thong segment fastener attached thereto proximate to the stirrup and the thong segment fastener is configured to engage with the fastener on the thong.

10. The protective prosthetic foot shell cover of claim 9 wherein the fastener attached to the thong and the thong segment fastener are one of a generic hook and loop type fastener, Velcro™, and a snap.

11. The protective prosthetic foot shell cover of claim 1 further including a sandal portion integrally attached to the stirrup and configured to be spaced from top portion.

12. The protective prosthetic foot shell cover of claim 11 wherein the shell cover is for use with a sandal having at least one fastener attached thereto and wherein the sandal portion further includes sandal at least one fastener attached to sandal portion and the sandal fastener is configured to engage with the fastener on the sandal.

13. The protective prosthetic foot shell cover of claim 12 wherein the sandal includes a plurality fasteners and the sandal portion includes a plurality fasteners

14. The protective prosthetic foot shell cover of claim 12 wherein the at least one fastener attached to the sandal and the sandal at least one fastener are one of a generic hook and loop type fastener, Velcro™, and a snap.

15. The protective prosthetic foot shell cover of claim 1 wherein the top portion further includes a plurality of links for receiving a tie.

16. The protective prosthetic foot shell cover of claim 15 wherein the tie is tied to a shoe.

17. The protective prosthetic foot shell cover of claim 1 wherein the shell cover is customizable.
